(19) United States
(12) Patent Application Publication
(10) Pub. No.: US 2025/0384983 A1
(43) Pub. Date: Dec. 18, 2025

(54) PERSONALIZED CARE RECOMMENDATION USING GENETICS ANALYSIS

(71) Applicant: ELC MANAGEMENT LLC, Melville, NY (US)

(72) Inventor: Christopher Aidan, Austin, TX (US)

(21) Appl. No.: 18/743,404

(22) Filed: Jun. 14, 2024

(51) Int. Cl. G16H 20/10; G16H 10/40; G16H 10/60; G16H 50/20

(52) U.S. Cl. CPC G16H 20/10; G16H 10/40; G16H 10/60; G16H 50/20

(57) ABSTRACT

A system for providing a personal care recommendation, and techniques for generating personal skin care recommendations, are provided. Example systems may include one or more processors and a diagnostic device to detect a personal care condition. The processors can assess a genetic profile of a user of the system and predict changes in the personal care condition based on the genetic profile and on information provided by the diagnostic device. The processors can generate the personal care recommendation based on the personal care condition or predicted changes in the personal care condition. A user interface coupled to the one or more processors can provide the generated personal care recommendation to the user.

### FIELD OF THE INVENTION

The present invention relates generally to the field of personal care and, more specifically, to systems capable of providing skin care recommendations utilizing machine learning, artificial intelligence, augmented reality, and other technologies.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

With the advent of genomics, wearable diagnostic devices, and increased social media interaction, the personal products industry has ever-increasing access to consumer data. Nevertheless, consumers are left with little guidance in how to select products or implement personal care regimens that suit their personal genetics, lifestyle, and environment.

### SUMMARY

In one aspect, a system for personal care recommendations is provided, comprising: one or more processors; a diagnostic device coupled to the one or more processors, the diagnostic device configured to detect a personal care condition; one or more non-transitory memory devices storing computer-readable instructions that, when executed by the one or more processors, cause the one or more processors to: assess a genetic profile of a user of the system; predict changes in the personal care condition based on the genetic profile and on information provided by the diagnostic device; and generate the personal care recommendation based on at least one of the personal care condition or the predicted changes in the personal care condition; and a user interface coupled to the one or more processors, the user interface configured to provide the personal care recommendation to the user. The system may include additional, fewer, or alternate elements, including those discussed elsewhere herein.

In another, a computer-implemented method of providing a personal care recommendation is provided. The method may include assessing a genetic profile of a user; predicting changes in a personal care condition based on at least one of the genetic profile or an output of a diagnostic device; generating the personal care recommendation based on at least one of the personal care condition or the predicted changes in the personal care condition; and providing the personal care recommendation to the user. The method may include additional, fewer, or alternate actions, including those discussed elsewhere herein.

In still another aspect, a non-transitory computer-readable storage medium storing instructions for providing a personal care recommendation is provided. The computer-readable instructions, when executed by one or more processors, may cause the one or more processors to perform a method. The method may include assessing a genetic profile of a user; predicting changes in a personal care condition based on at least one of the genetic profile and an output of a diagnostic device; generating the personal care recommendation based on at least one of the personal care condition or the predicted personal care condition; and providing the generated personal care recommendation to the user. The instructions may direct additional, fewer, or alternative functionality, including that discussed elsewhere herein.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof.

There are shown in the drawings arrangements which are presently discussed, it being understood, however, that the present embodiments are not limited to the precise arrangements and instrumentalities shown, wherein:

FIG. 1 depicts an exemplary computer system for providing a personal care recommendation, according to some embodiments;

FIGS. 2A-2D depict examples of displays as may be provided by a user interface associated with a system for providing personal care recommendations, according to some embodiments; and

FIG. 3 depicts a flow diagram of an exemplary computer-implemented method for providing personal care regimens, according to some embodiments.

While the systems and methods disclosed herein are susceptible of being embodied in many different forms, they are shown in the drawings and are described herein in detail specific exemplary embodiments thereof, with the understanding that the present disclosure is to be considered as an exemplification of the principles of the systems and methods disclosed herein and is not intended to limit the systems and methods disclosed herein to the specific embodiments illustrated. In this respect, before explaining at least one embodiment consistent with the present systems and methods disclosed herein in detail, it is to be understood that the systems and methods disclosed herein are not limited in its application to the details of construction and to the arrangements of components set forth above and below, illustrated in the drawings, or as described in the examples.

Methods and apparatuses consistent with the systems and methods disclosed herein are capable of other embodiments and of being practiced and carried out in various ways. Also, it is to be understood that the phraseology and terminology employed herein, as well as the abstract included below, are for the purposes of description and should not be regarded as limiting.

### DETAILED DESCRIPTION

### Overview

The present disclosure provides a personalized personal care regimen (in some examples directed to skincare and beauty, although embodiments are not limited thereto) that can leverage artificial intelligence (AI), machine learning (ML), genetic data, diagnostic device data, lifestyle factors, social media inputs, geographical information, and other relevant inputs. The system collects and processes these data to create the personal care regimen. Systems according to embodiments may identify trends (using e.g., social media, product sales databases, print media and other traditional media, etc.) and may adjust the personal care regimen or provide further insights or features regarding the personal care regimen based on the trends. Systems can also predict future skin conditions using diagnostic devices and predictors based on lifestyle and genetic factors. Systems according to some embodiments can ensure product safety and product efficacy using feedback provided through machine learning or other mechanisms.

The system described herein can integrate a goal-setting system, a predictive skincare system, and a notification system to increase user interaction and engagement, which in turn can lead to greater compliance with a skin care regimen. The system can use a diagnostic device to detect a current personal care (e.g., skin care) condition, and then uses ML, AI, AR, VR, and other technologies to display possible future skin care conditions based on likely, actual, or predicted skin care product usage. The system may collect, retrieve, or be provided with analysis of genetic data from the user and analyzes the genetic data to identify genetic markers that could impact skin health, beauty product efficacy, and potential allergic or adverse reactions to ingredients. The system may provide an interface to social media to collect information provided in social media posts, to detect trends and user sentiment. The social media interface can help the system to foster of a sense of community among users with similar genetic makeups, enable users to connect, share experiences, and provide advice. The system can determine lifestyle habits through direct user input or through wearable devices to detect factors that could impact skin health.

The system may include a user interface, which allows the user to interact with the system, view their personalized regimen, track their progress over time, and adjust their skincare goals as needed. The system may also include a personalized notification system, which sends reminders to the user to apply products, make lifestyle changes, etc., based on knowledge of user schedules and routines. User privacy is protected using a blockchain-based data storage module for secure, transparent, and immutable storage of genetic data, diagnostic device data, and the personalized beauty regimen.

### Example System

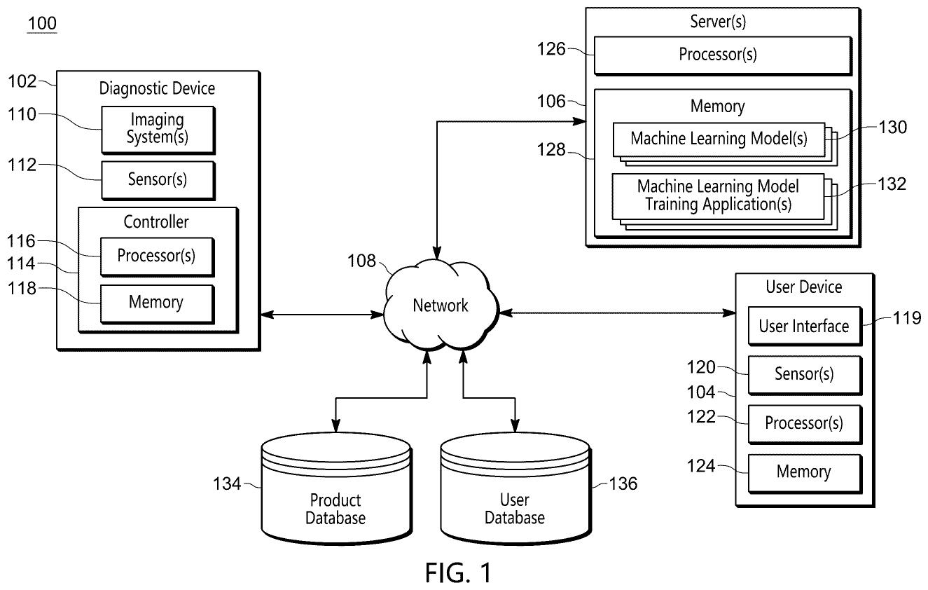

FIG. 1 depicts an exemplary computer system 100 for personalized care regimen, according to one embodiment. The high-level architecture illustrated in FIG. 1 may include both hardware and software applications, as well as various data communications channels for communicating data between the various hardware and software components, as is described below.

The system 100 may include a diagnostic device 102 as well as, in some cases, one or more user computing devices 104 (which may include, e.g., smart phones, smart watches or fitness tracker devices, tablets, laptops, virtual reality headsets, smart or augmented reality glasses, wearables, etc.), and/or one or more server(s) 106. The diagnostic device 102, user device(s) 104, and/or server(s) 106 may be operable to communicate with one another via a wired or wireless computer network 108, and/or via short range signals, such as BLUETOOTH signals. In some example embodiments, the diagnostic device 102 (or one or more components thereof) can be included within user device(s) 104.

Although one diagnostic device 102, one user device 104, one server 106, and one network 108 are shown in FIG. 1, any number of such diagnostic devices 102, user devices 104, servers 106, and networks 108 may be included in various embodiments. To facilitate such communications, the diagnostic device 102, user devices 104, and/or servers 106 may each respectively comprise a wireless transceiver to receive and transmit wireless communications.

The diagnostic device 102 is configured to detect a personal care (e.g., skin care) condition. The diagnostic device 102 can include one or more imaging system/s 110 to detect a skin care condition (e.g., skin conditions such as sun damage, acne, redness, dryness, hyperpigmentation, eczema, allergic reactions, and the like although embodiments are not limited thereto) and at least one sensor 112 for scanning the user's skin or detecting moisture, oil, and the like. The sensors 112 may include other types of sensors operable to capture biometric data associated with the user, such as facial recognition data, fingerprint recognition data, iris recognition data, etc. The sensors 112 can also include thermal sensors, liquid sensors, and the like. Any or all of the imaging system 110 and sensor(s) 112 can be housed separately from each other and/or from a main diagnostic device 102, or in groups of similar imaging system 110 types or sensor(s) 112 type. Any or all of the imaging system 110 and sensor(s) 112 can be provided in a wearable device (e.g., sweat monitor, heart rate monitor, smart watch, clothing, etc.). One or more imaging system 110 or sensor(s) 112 can be used to perform (or to provide inputs for) spectral analysis of the user's skin, surface of the skin, products on or near the skin, and the like.

The diagnostic device 102 transmits images (photographs, video, etc.) and sensor measurements to other components of the diagnostic device 102 (e.g., the controller 114) or to other components of the system 100 (e.g., the server 106). The controller 114 can include one or more processor(s) 116, as well as one or more computer memories 118. The controller 114 can analyze images for uneven color, pigmentation, and the like. The controller 114 can detect or determine measurements, such as distance between eyes, length of chin, and the like based on the image(s). The controller 114 can use the sensor measurements to detect oil and moisture saturation of the user's skin, reactivity of the user's skin to a specific substance, or any other condition. Any or all of the above controller 114 functions can additionally or alternatively be performed in other components of the system 100 (e.g., the server 106 or user device 104 or any other device not shown connectable through the network 108).

The imaging system 110 can capture image(s) of the user's skin at one or more points in time so that the server 106 (or components thereof) can perform time-based analysis of the effectiveness of products, changes due to time of year, and the like. Similarly, sensors 112 can capture and provide measurements at one or more points in time for similar time-based analysis or change analysis. As described later herein, components of the system 100 can use images, measurements, etc. in machine learning algorithms or other processing to perform predictions, provide product recommendations, and the like. The controller 114 can control the imaging system 110, sensors 112 to take periodic measurements/images or on-demand measurements and images based on requests from the server 106, on specific programming of the controller 114, based on user request, or the like.

The memories 118 may include one or more forms of volatile and/or non-volatile, non-transitory, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memories 118 may store an operating system (OS) (e.g., iOS, Microsoft Windows, Linux, UNIX, etc.) capable of facilitating the functionalities, apps, methods, or other software as discussed herein.

Generally speaking, the memories 118 may store instructions that, when executed by the processor(s) 116, cause the processors 116 to receive images from the imaging system 110, and measurements from the sensors 112. The memories 118 can cause the controller 114 to control image capture schedules, sensor measurement schedules, and the like and to encode messages for communicate to the network 108 or to the server 106.

Furthermore, the memories 118 may store instructions that, when executed by the processor(s) 116, cause the processor(s) 116 to analyze images associated with skin care products or treatments to identify particular products or characteristics thereof. For instance, the memories 118 may store instructions that, when executed by the processor(s) 116, cause the processor(s) 116 to capture image data associated with packaging of various skin care products or treatments and analyze the image data associated with the packaging of the various skin care products or treatments to identify the respective products/treatments based on their packaging. The identification can be provided for updating user schedules, notifying of possible product formula updates, and the like.

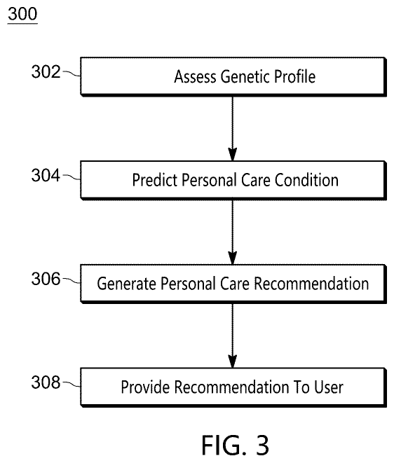

Furthermore, in some examples, the instructions stored on the memories 118 may cause the processor(s) 116 and/or the controller 114 to perform any or all of the steps of the method 300 discussed below with respect to FIG. 3.

The user device 104 includes a user interface 119 operable to receive inputs and selections from the user of the system 100 (e.g., the end user or customer), and/or to provide audible or visual feedback to the user.

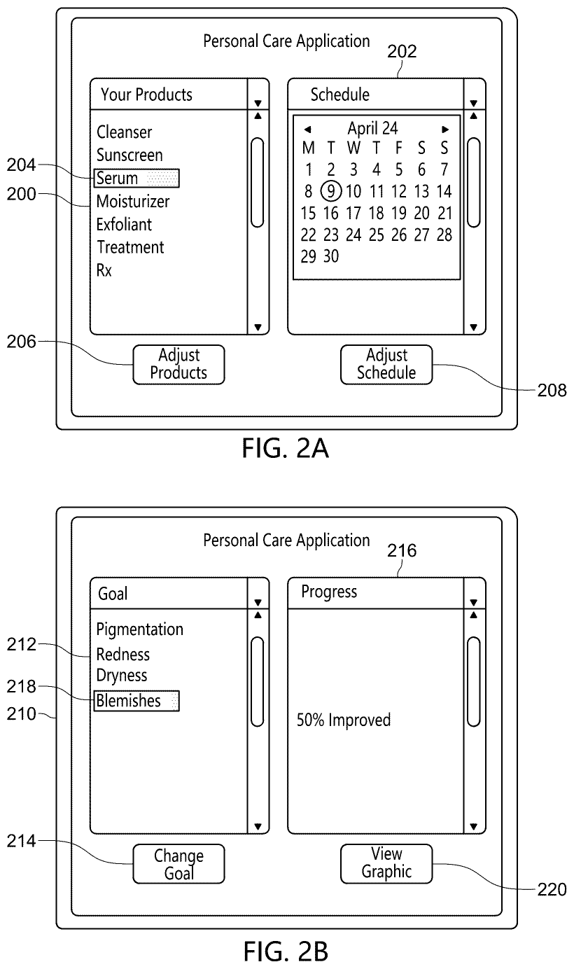

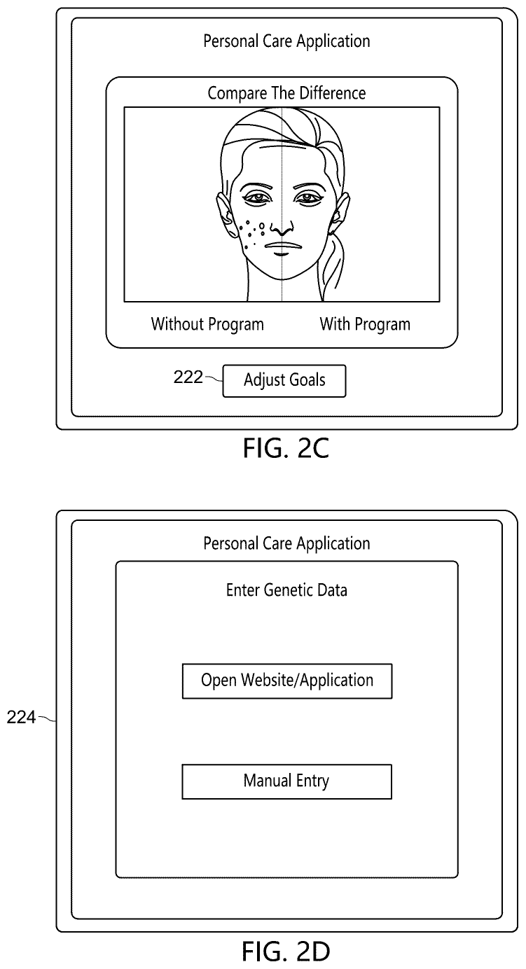

For instance, the user interface 119 may provide interactive displays via which users allows the user to interact with the system, view their personalized regimen, track their progress over time, and adjust their skincare goals as needed, as described later herein with respect to FIGS. 2A-2C. The user interface 119 can also provide alarms, alerts, and the like that reminds the user to apply products, make lifestyle changes such as increasing sleep, improving hydration, reducing or eliminating alcohol/tobacco use, and the like. The reminders can be based on calendar data or other data indicating user routine so that the user is reminded to use products at a proper time of day. The proper time of day can be predicted using machine learning models 130 described in more detail later herein based on inputs from wearable devices, user location, etc.

The user may also use the user interface 119 to provide an image or a social media link. The image can be used similarly to the image described above and the social media link can be used to retrieve periodic image data or past image data, or for other purposes such as community support, user sentiment detection, and other features as described later herein.

In some examples, the user interface 119 may further include an augmented reality (AR) component operable to generate and display an AR rendering of three-dimensional map of the user's face. In some cases, the AR rendering may be overlaid upon an image or video of the user's face as captured in real-time by any of the sensors 112 provided in the diagnostic device 102. The AR technology can also be used to provide users with a visual simulation of potential future skin conditions based on their personalized beauty regimen.

Moreover, in some examples, the user interface 119 may be operable to receive feedback from a user. For example, one group of users or type of users may provide feedback that they felt a moisturizer was too thick, or that the moisturizer caused breakouts. Manufacturers could react by changing the moisturizer formulation, or advertising to a different demographic of customer (e.g., to older customers more likely to have dry skin or less likely to experience breakouts), for example. The feedback can be provided to machine learning algorithms to improve predictions, product recommendations, regimen recommendations and the like by analyzing patterns in user feedback. The feedback could also be analyzed by other types of software programs/modules to detect whether a certain type of user or demographic of user is more likely to complain about certain types of products, to improve recommendations made to similar users. Feedback can include automated or user-independent feedback capture including analyzing text reviews for sentiment, categorizing feedback into different themes, and identifying common issues or praises.

The user device 104 may include, or may be operable to communicate with, the user interface 119 as described earlier herein. Furthermore, the user device 104 may include, or may be operable to communicate with, one or more respective sensors 120, which may include similar sensors and/or sensor functionality as discussed above with respect to the sensors 112 of the diagnostic device 102. The sensors 120 can further include visual-based sensors, such as cameras or video recorders, which the user device 104 can provide to the diagnostic device 102 or to the server 106 for processing and analysis as described earlier herein.

Moreover, the user device 104 may include one or more processor(s) 122, as well as one or more computer memories 124. Memories 124 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memories 124 may store an operating system (OS) (e.g., iOS, Microsoft Windows, Linux, UNIX, etc.) capable of facilitating the functionalities, apps, methods, or other software as discussed herein. The memories 124 may store instructions that, when executed by the processor(s) 122, cause the processor(s) 122 to receive input from a user as provided via the user interface 119 and send the received user input to the diagnostic device 102 (e.g., via the network 108) and/or to the server 106, in some cases responsive to a request for such user input from the diagnostic device 102. Furthermore, in some examples, the memories 124 may store instructions that, when executed by the processor(s) 122, cause the processor(s) 122 to capture sensor data via one or more sensors 120, in some cases responsive to a request for particular sensor data from the diagnostic device 102, and may send the captured sensor data to the diagnostic device 102.

Furthermore, in some examples, the instructions stored on the memories 124 may cause the processor(s) 122 to perform any or all of the steps of the method 300 discussed below with respect to FIG. 3.

In some embodiments the server 106 may comprise one or more servers, which may comprise multiple, redundant, or replicated servers as part of a server farm. In still further aspects, such server(s) 106 may be implemented as cloud-based servers, such as a cloud-based computing platform. For example, such server(s) 106 may be any one or more cloud-based platform(s) such as MICROSOFT AZURE, AMAZON AWS, or the like. Such server(s) 106 may include one or more processor(s) 126 (e.g., CPUs) as well as one or more computer memories 128.

The memories 128 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memories 128 may store an operating system (OS) (e.g., Microsoft Windows, Linux, UNIX, etc.) capable of facilitating the functionalities, apps, methods, or other software as discussed herein.

Additionally, or alternatively, the memories 128 may store product data, including product identifiers and ingredients, which can be updated by product manufacturers in real-time. Product data may also be stored in a product database 134 (or in multiple such databases), which may be accessible or otherwise communicatively coupled to the server 106. The user data may include previous products used by the user, user preferences, and various other data associated with the user, and may also be stored in a user database 136 (or in multiple such databases), which may be accessible or otherwise communicatively coupled to the server 106. Furthermore, in some examples, the product data and the user data may be stored in the same database, which may be accessible or otherwise communicatively coupled to the server 106.

Furthermore, the memories 128 may store instructions that, when executed by the processors 126, cause the processors 126 to receive data from various databases such as the databases 134 and 136, and/or data from the diagnostic device 102 and/or the user device 104 (e.g., via the network 108). The data from the diagnostic device 102 and/or the user device 104 may include, for instance, data captured by the sensors 112 or imaging system 110 of the diagnostic device 102 and/or data captured by the sensors 120 of the user device 104, data input by a user via a user interface 119 of the user device 104, etc. The instructions stored on the memories 128, when executed by the processors 126, may cause the processors 126 to analyze data received from the database, and/or the diagnostic device 102 and/or the user device 104 to make a recommendation or prediction based on the received data, and subsequently send the recommendation and/or prediction to the diagnostic device 102 and/or the user device 104. For instance, this analysis and recommendation and/or prediction may be based upon applying a trained machine learning model 130 to the data received from the databases and/or the diagnostic device 102 and/or the user device 104.

The memories 128 may store one or more machine learning models 130, and/or one or more respective machine learning model training applications 132 and the processor(s) 126 can execute or implement machine learning models 130 and machine learning model training applications 132. These machine learning models 130 may include, for instance, a machine learning model trained to analyze genetic data, diagnostic device 102 data, lifestyle factors, social media inputs, geographical information, and other relevant input data to generate a personal care (e.g., skin care or beauty care) regimen for a user of the system 100. The machine learning model can output trend information or data, forecast future skin conditions of the user based on any of the above-described inputs. Other software applications or modules can provide updated product safety information and product efficacy information based on new manufacturer information and scientific discoveries or based on user feedback (whether directly input or inferred from social media or the like). As such, by implementing or executing the machine learning models 130, the processor 126 can assess a genetic profile of a user of the system; predict changes in the skin or other personal care condition based on the genetic profile and on information provided by the diagnostic device 102; and generate the personal care recommendation based on the predicted personal care condition. Example changes could include changes common to persons of similar genetics, e.g., hyperpigmentation, tendency for reduced elasticity or wrinkling, acne, and the like. Example personal or skin care recommendations can include a recommendation to include an exfoliant or moisturizer in the user's skin care regimen, the recommendation to use a particular type of cleanser, a schedule for applying any type of product, and the like.

The server 106 can use the machine learning models 130 or other software program or module to track and analyze the impact of seasonal changes on skin health, taking into consideration factors such as humidity, temperature, and sunlight exposure. The machine learning models 130 adjust the personalized beauty regimen accordingly to optimize skin health in different seasons or other software programs/modules can determine or retrieve expected correlations of skin care conditions to these or similar seasonal changes. The machine learning models 130 can be trained to provide predicted outputs based on the influence of geographical location and local environmental factors on skin health. The machine learning models 130 can output or update product recommendations, product application schedules, and the like based on this geographical data to best suit the local environment. The machine learning models 130 can include models such as decision trees, support vector machines, neural networks, and the like.

The server 106 can use the machine learning models 130 or other software programs or modules to identify correlations between genetic markers and skin health. The machine learning models 130 use these correlations to predict how a user's skin may respond to different beauty products and treatments, or other software programs/modules can retrieve expected responses from a database or other data storage. The machine learning models 130 can output or update product recommendations, product application schedules, and the like based on the genetic information. Inputs can be additionally provided from known or detected family members and predictions made regarding likely effects on a user based on product effects on a family member. Predictions can include predictions of potential allergic or adverse reactions based on the user's genetic data or based on user knowledge of same or similar products to which the user has had an adverse reaction in the past. Outputs of the models 130 or other software programs or modules therefore can include adjustments to recommendations and personalized regimens based on problematic skin care ingredients.

The server 106 can use the machine learning models 130 or other software programs/modules to track trends in the skincare and beauty industry. By analyzing social media data and other digital sources, the system 100 can apply outputs of software programs/modules to provide information to users about new developments that may benefit their personalized beauty regimen. The machine learning models 130 can also be used to analyze patterns in user feedback. This includes analyzing text reviews for sentiment, categorizing feedback into different themes, and identifying common issues or praises. Insights derived from this feedback analysis are used to improve the system 100 and the personalized beauty regimen. Feedback analysis can also include predictions or analysis of user engagement with their personal care regimen. For example, factors such as frequency of product application, response to product recommendations, and adherence to the regimen can be provided as inputs to the machine learning models 130 to make a prediction or recommendation of strategies to improve consistency and engagement.

The system 100 can use machine learning models 130 or other software programs/modules to analyze data from social media and other digital platforms to determine the potential impacts of emotional health and stress levels on skin health. The system 100 then adjusts the beauty regimen based on this data, adding products or treatments according to predicted outputs of the machine learning models 130 to help alleviate skin conditions caused or exacerbated by stress.

The system 100 can use machine learning models 130 to predict future skin conditions based on genetic and lifestyle data, as well as data from the diagnostic device 102. Some diagnostic device 102 data may also indicate loss of product efficacy, and that information can be used to adjust product recommendations. The machine learning models 130 can provide predicted outputs to the system 100 so that preemptive skincare treatments and adjustments to the beauty regimen can be made. Outputs can also be provided directly or with further processing to the user interface 119 for further motivating the user.

In some examples, one or more machine learning model(s) 130 may be executed on the server 106, while in other examples one or more machine learning model(s) 130 may be executed on another computing system, separate from the server 106. For instance, the server 106 may send data to another computing system, where a trained machine learning model 130 is applied to the data, and the other computing system may send a prediction or recommendation, based upon applying the trained machine learning model 130 to the data, to the server 106. Moreover, in some examples, one or more machine learning model 130 (s) may be trained by respective machine learning model training application(s) 132 executing on the server 106, while in other examples, one or more machine learning model(s) 130 may be trained by respective machine learning model training application(s) executing on another computing system, separate from the server 106.

Whether the machine learning model(s) are 130 trained on the server 106 or elsewhere, the machine learning model(s) 130 may be trained by respective machine learning model training application(s) 132 using training data (including historical data in some cases), and the trained machine learning model(s) 130 may then be applied to new/current data that is separate from the training data in order to determine, e.g., predictions and/or identifications related to the new/current data.

For example, a machine learning model 130 trained to analyze data associated with a personal care regimen may be trained by a machine learning model training application 132 using training data including genetics of multiple (e.g., hundreds or thousands) of users or of an entire regional population, geographical information, a history of products successfully used by that group of users, and other relevant inputs. For example, products that were successfully used by a group of users having a particular genetic profile may have resulted in positive changes to the users' skin health, either subjectively as reported by the users or as measured by skin care practitioners or devices. The machine learning model 130 can therefore be trained to learn which products or product types should be recommended for users of similar genetics. As another example, products that were successfully used by a group of users in a geographic location may have resulted in positive changes to the users' skin health, either subjectively as reported by the users or as measured by skin care practitioners or devices. The machine learning model 130 can therefore be trained to learn which products or product types should be recommended for users in that geographical region or regions of a similar climate.

As another example, a machine learning model 130 trained to provide a personalized care regimen may be trained by a machine learning model training application 132 using training data that includes images of multiple users. The images are labeled with regimens for each user and an indication or evaluation as to whether the skin care regimen was beneficial. Once sufficiently trained using this training data, such a machine learning model 130 may be applied to a new person, a new image of the same person or a different person, etc., such as an image provided by a user via a user interface 119, or an image from a social media, and the machine learning model 130 can identify or predict personal care products for the new person or based on the new image, that would be beneficial based on the learning.

As another example, a machine learning model 130 trained to predict facial aging of a user may be trained by a machine learning model training application 132 that includes images of multiple users at various times in their lives. The images can be labeled with information as to location of any wrinkles or other age indicators, level of wrinkles, and the like. Once sufficiently trained using this training data, such a machine learning model 130 may be applied to predict how someone of a similar genetic background, ethnic group, etc. will age.

As another example, a machine learning model 130 trained to predict how a product will affect a user's face over time may be trained by a machine learning model training application 132 that includes images of multiple users as those users have used the products over a period of time (e.g., training data will include multiple images of the users spanning a time frame). The images can be labeled with information as to how each product affected the users' appearance. Once sufficiently trained using this training data, such a machine learning model 130 may be applied to predict how each product will affect the user's face over time.

Moreover, as another example, a machine learning model 130 trained to provide a personalized care regimen can be trained by a machine learning model training application 132 using training data including images or other sensor data provided by the diagnostic device 102 and associated with various individuals' skin, and indications of skin types, skin health conditions, or other skin characteristics associated with the various individuals' skin. For instance, images of individuals having various skin types may be labeled with the respective skin types shown in each image. Similarly, images of individuals having various skin health conditions may be labeled with an indication of the health condition, the location of visual indicators associated with the health condition shown in the image, etc. Furthermore, images of individuals having various genetic traits may be labeled with the respective genetic traits. These labeled images may be used as training data, and once sufficiently trained using this training data, such a machine learning model 130 may be applied to a new image, video, or the like associated with a user's face (e.g., an image or video captured by the sensors 112, 120, etc., in real-time), and may identify/predict a skin type, skin health condition, genetic condition and/or other skin characteristic associated with the user's face. The skin type or health condition can be matched with products or formulations known to be beneficial to that skin type/condition/genetics, either as learned by the machine learning model 130 or as stored in lookup tables or other databases. The server 106 can provide a personalized care regimen based on the machines.

Additionally, as another example, a machine learning model 130 trained to provide personalized care regimens may be trained by a machine learning model training application 132 using any updated training data based on user feedback, product formulation changes, new product availability, and the like. Recommendations can be updated by other types of software applications or modules based on scientific discoveries, changes in the user's skin as captured by the diagnostic device 102 or user device 104, location data or geographical changes pertaining to the user or similar users, etc. The machine learning model 130 may be trained by a machine learning model training application 132 using training data including products selected by previous users, characteristics of the previous users, input/feedback from the previous users about the products, etc. For instance, various products may be labeled with indications of characteristics of users who gave positive feedback regarding the products, indications of similar products receiving positive or negative feedback, etc. Once sufficiently trained using this training data, such a machine learning model 130 may be applied to a user, the user's characteristics, and previous care products selected/liked by the user and may predict/suggest other products that the user may enjoy.

In various aspects, the machine learning model(s) 130 may comprise machine learning programs or algorithms that may be trained by and/or employ neural networks, which may include deep learning neural networks, or combined learning modules or programs that learn in one or more features or feature datasets in particular area(s) of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques.

In some embodiments, the artificial intelligence and/or machine learning based algorithms used to train the machine learning model(s) 130 may comprise a library or package executed on the server 106 (or other computing devices not shown in FIG. 1). For example, such libraries may include the TENSORFLOW based library, the PYTORCH library, and/or the SCIKIT-LEARN Python library.

Machine learning may involve identifying and recognizing patterns in existing data (such as training a model based upon historical data) to facilitate making predictions or identification for subsequent data (such as using the machine learning model on new/current data order to determine a prediction or identification related to the new/current data).

Machine learning model(s) may be created and trained based upon example data (e.g., “training data”) inputs or data (which may be termed “features” and “labels”) to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based upon the discovered rules, relationships, or model, an expected output.

In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated. The disclosures herein may use one or both of such supervised or unsupervised machine learning techniques.

In addition, memories 128 may also store additional machine-readable or computer-readable instructions, including any of one or more application(s), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For instance, in some examples, the computer-readable instructions stored on the memory 128 may include instructions for carrying out any of the steps of the method 300 via an algorithm executing on the processors 126, which is described in greater detail below with respect to FIG. 3. It should be appreciated that one or more other applications may be envisioned and that are executed by the processor(s) 126. It should be appreciated that given the state of advancements of mobile computing devices, any or all of the processes functions and steps described herein may be present together on a mobile computing device, such as the user device 104, or the diagnostic device 102.

### Example User Interface Displays

FIGS. 2A-2D depict exemplary user interface displays as may be provided by a user interface for a user of the system 100 (e.g., a user interface 119 of the user device 104). In some embodiments, certain displays or depictions of the personal care regimen, before/after images, 3D images, etc. can be provided in a separate device such as another user device similar to the user device 104 (e.g., a second smartphone or tablet, desktop computer, laptop, etc.)

FIG. 2A illustrates an example user interface display via which a user may view products recommended in a personal care regimen. List 200 includes a list of one or more products currently within the recommended personal care regimen. The list 200 can include brand names or categories and can be updated as the personal care regimen is updated. The display can include details on a schedule 202 for using a selected product 204. The schedule 202 can include dates, intervals, time of day, and other information. Various other interface items 206, 208 can be included. For example, a user can adjust products using interface item 206.

The system 100 can automatically populate the list 200 with recommended products. In addition, the user can manually add products to the list 200. The products in the list 200 can be automatically updated by the system 100 when machine learning algorithms or other components of the system 100 determine that products have changed, or that the recommendation is to be changed, or that the product list is to be changed. The interface item 206 allows users to manually change items in the regimen. Users could also adjust or add details about the products in the list 200, including dates purchased, where bought, etc. Similarly, a user can adjust schedules for product use with interface item 208.

FIG. 2B illustrates an example user interface display 210 via which a user may manage goals in his or her personal care regimen. List 212 includes a list of one or more goals of the recommended personal care regimen. The list 212 can include any number of typical goals or the user can enter, change, or add detail regarding goals using interface item 214. The list 216 can include details on progress with respect to selected goal 218. The user can request to view progress in a graphic using interface item 220.

FIG. 2C illustrates an example graphic of the user or similar user's skin. The graphic can include views of the user's skin if the user were not using the program, side by side or conjoined with a view of the user's skin if the user were using the program. The graphic shown in FIG. 2C can display the face or a facial feature of the user or of a user having similar genetics, skin tone, etc. The processor 126 or other component of the server 106 can adapt images provided by the user or automatically retrieved from social media, a user digital photo album, and the like to generate “before and after” images or predictive images. The processor 126 or other component of the server 106 can use photographic aging software or predictions based on increased or decreased product use, a worsening or improvement in certain skin conditions, or the facial aging machine learning model described above to generate various versions of images of the user.

FIG. 2C can depict any percentage of compliance with the program. The view can be two-dimensional or three-dimensional and can include actual images of the user or modified/enhanced views and images. One or more user interface items 222 can be provided to allow the user to view him- or herself with modification of the goals, modification in compliance, etc. In some examples, the preview may be a generalized preview, e.g., illustrating examples of other individuals to whom the regimen has been applied, or illustrating examples of a three-dimensional rendering of the look as applied to a three-dimensional model of a face.

FIG. 2D illustrates an example user interface display 224 via which a user may provide genetic data, which may or may not be relevant to his or her personal care regimen as described earlier herein. The user can choose to open a website, application, or other system to download genetic profile information, or manual entry can be selected. The user can provide, or may be asked to provide, permission to access his or her genetic profile or other genetic information. For example, certain regulatory or agency rules or data privacy laws may require that user permission be requested before accessing personal data such as genetic data or other sensitive data. Permission can be requested or granted through, for example, terms or conditions provided to the user at time of installation of the personalized skin care application, or periodically, etc. Additionally or alternatively, permission can be granted on an opt in or an opt out basis. For example, the user may be assumed to not grant permission to access certain types of data unless he or she opts in, or the user may be assumed to grant permission to access certain types of personal data unless the user opts out.

### Example Method

FIG. 3 depicts a flow diagram of an exemplary computer-implemented method for providing a personal care recommendation, according to one embodiment. One or more operations of the method 300 may be implemented as a set of instructions stored on a computer-readable memory (e.g., memory 118, memory 124, memory 128, etc.) and executable on one or more processors (e.g., processor 116, controller 114, processor 122, processor 126, etc.).

The method 300 may begin with operation 302 with assessing a genetic profile of a user of the system 100. In some examples, operation 302 can include collecting genetic data from the user. In examples, this can be done through a user interface such as shown in FIG. 2D. In some examples, collecting can be done by receiving data or analysis related to a deoxyribonucleic acid (DNA) sample of the user. For example, data or analysis can be retrieved from or provided by a genetics testing service, ancestry research organization/service, and the like. In still other examples, collecting includes accessing a genetic testing service (e.g., by the user accessing a service website as described with respect to FIG. 2D, or with permission of the user, etc.). The assessing can also include identifying genetic markers of the user, where genetic markers indicate conditions that affect one or more of skin health, skin care product efficacy, or potential allergic reactions to skin care products.

In some examples, the method 300 can include predicting the personal care condition using a trained machine learning model that is trained using genetic profiles of a population. The model can be updated using expanded training data of a second population that is a superset of the initial population. For example, once a machine learning model is trained using an initial geographic population or group of users having same or similar genetics, the machine learning model can be trained again or updated using a larger geographic population or group that may include at least the initial geographic population or group of users. By way of illustration, an initial population can include a number of people in a town or other geographic region, and the second population can include more people within that town, or people across a broader geographical region. The model can also be updated or trained to predict personal care conditions that are prevalent among one or more of an ethnic group, a cultural group, or a national group.

Further, predicting the personal care condition can include requesting or receiving input pertaining to lifestyle information, diet information, environmental information pertaining to the user or a location of the user. As such, the method 300 can include connecting with any other remote or local device or application that could include these inputs, including other health-based applications or devices, weather applications, and the like. For example, any or all of the above inputs could be received from a wearable device or an Internet of Things (IoT) device proximate the user.

Predicting and updating predictions can be further based on data retrieved from a social media network and accordingly the method 300 can include accessing a user profile on a social media network, either automatically through any of the network connections described herein, or through direct user input/linking. The method 300 can further include sharing the personal care recommendation or data pertaining thereto, with the social media network

The method 300 can continue with operation 304 with predicting changes in a personal care condition based on the genetic profile and on information provided by a diagnostic device (e.g., diagnostic device 102). This predicting can be done using a trained machine learning model (e.g., as described above with reference to elements 130, 132). As described earlier herein, inputs to the machine learning model can include skin care product data. Updates to the trained machine learning model 130 can be based on feedback input by the user, among other modes, inputs or methods of updating. Feedback can include reviews of products, comments on products or properties thereof, statements regarding whether products had desired effects on the user's skin, and the like. The feedback can be provided in the form of end-user actions to retrain and improve the corresponding machine learning model by helping the machine learning model determine whether previous learning was in error.

The method 300 can continue with operation 306 with generating a personal care recommendation based on the predicted personal care condition. The personal care recommendation can include a product recommendation, lifestyle change recommendation (e.g., recommendations regarding diet, hydration, exercise, and the like), etc. When a product is recommended, the product recommendation can include a recommendation of at least one skin care product. In some example embodiments, the system 100 may provide access to an online purchasing system to purchase a product associated with the product recommendation. The product recommendation may be generated to account for a user preference for cruelty-free products, organic products, vegan beauty products, and the like.

The personal care regimen can include a recommendation for timing, scheduling, sequence, etc. for application of personal care operations. For example, a user may be instructed to apply serum only once per week, and only after washing his or her face.

The method 300 can continue with operation 308 with providing the personal care recommendation to the user. In examples, the providing can be through a user interface such as described with respect to FIGS. 2A-2D. The method 300 can further include providing access to further information, such as science information, chemistry information, or expert advice, pertaining to the recommendation. This can be accessed through the user interface. The user interface can additionally include an alert system to alert the user to product updates of products related to the personal care recommendation, or to alert the user to effects of non-compliance, scheduling alerts, or any other audible or visual alert.

In embodiments in which sensors are provided within multiple devices, systems, or apparatuses, at least one device can request sensor data from, and/or receive sensor data captured by, the sensors of the separate device via a communication interface, e.g., via a network (e.g., network 108), via a short-range signal between the separate device/s, and/or via a wired connection between device/s.

In some examples, one or more of the processor/s can store diagnostic device measurement data or genetic information in a user database. Methods described herein can further include providing encryption and security algorithms to protect user privacy, product manufacturer security, and the like.

The method 300 may further include using an imaging system configured to detect skin conditions. Spectral analysis or other analysis can be utilized to detect skin conditions. One or more diagnostic devices 102 or imaging systems can provide information regarding detected skin conditions as updated data, and the one or more processors, can execute updated training of the trained machine learning model using the updated data.

In some examples, the method 300 may include providing user instructions, guidance, support, tutorials, etc., associated with the personal care recommendation or any of the products recommended therein. Guidance can be provided at the user device 104, or by sending the user instructions, guidance, support, tutorials, etc. to a separate device to be presented via a user interface of the separate device.

In some examples, the method 300 may further include analyzing the sensor data in real-time to identify blemishes of the skin of the user, and automatically adjusting a product recommendation to a product to deal with more immediate needs (e.g., as opposed to long-term skin care goals). Machine learning algorithms and models can be updated accordingly, if it is or can be suspected that an allergic reaction has taken place or that a product is otherwise unsuitable for the user's skin. Additionally, the method 300 can include automatically generating alerts or notifications based on any identified skin reactions. For instance, the method 300 may include presenting such generated alerts via the user device 104, or to a separate device, wearable device, etc.

### ADDITIONAL CONSIDERATIONS

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” or “some embodiments” means that a particular element, feature, structure, or characteristic described in connection with the embodiment is included in at least one embodiment. The appearances of the phrase “in one embodiment” or “in some embodiments” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present), and B is false (or not present), A is false (or not present), and B is true (or present), and both A and B are true (or present).

In addition, use of “a” or “an” is employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system for providing personal care recommendations, diagnostic devices, user interface devices for displaying recommendations, and/or systems, methods, and/or techniques associated therewith. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

### ASPECTS

1. A system for providing a personal care recommendation, the system comprising: one or more processors; a diagnostic device coupled to the one or more processors, the diagnostic device configured to detect a personal care condition; one or more non-transitory memory devices storing computer-readable instructions that, when executed by the one or more processors, cause the one or more processors to: assess a genetic profile of a user of the system; predict changes in the personal care condition based on the genetic profile and on information provided by the diagnostic device; and generate the personal care recommendation based on at least one of the personal care condition or the predicted changes in the personal care condition; and a user interface coupled to the one or more processors, the user interface configured to provide the generated personal care recommendation to the user.

2. The system of aspect 1, wherein the one or more processors are configured to assess the genetic profile of the user by: collecting genetic data from the user; and identifying genetic markers of the user.

3. The system of aspect 2, wherein collecting genetic data comprises receiving data associated with deoxyribonucleic acid (DNA) sample of the user.

4. The system of any of aspects 2-3, wherein collecting genetic data comprises accessing a genetic testing service.

5. The system of any of aspects 2-4, wherein collecting the genetic data includes providing an encryption system to protect privacy of the genetic data.

6. The system of any of aspects 2-5, wherein the genetic markers indicate conditions that affect one or more of skin health, skin care product efficacy, or potential allergic reactions to skin care products.

7. The system of any of the preceding aspects, wherein one or more processors are configured to predict the personal care condition using a trained machine learning model.

8. The system of aspect 7, wherein the trained machine learning model is trained to identify at least one skin care product recommended for a user using training data including genetic profiles of a population labeled with the skin care products used by members of the population, wherein the one or more processors are configured to identify that the user has at least one characteristic in common with the population or is a member of the population.

9. The system of aspect 8, wherein the one or more processors are further configured to update the trained machine learning model using expanded training data of a second population that is a superset of the population wherein the one or more processors are configured to identify that the user has at least one characteristic in common with the second population or is a member of the second population.

10. The system of any of aspects 7-9, wherein the one or more processors are further configured to update the trained machine learning model with global datasets to predict personal care conditions that are prevalent among one or more of an ethnic group, a cultural group, or a national group.

11. The system of any of aspects 7-10, wherein the one or more processors are further configured to update the trained machine learning model based on feedback input by the user.

12. The system of any of aspects 7-11, wherein the one or more processors are configured to store diagnostic device measurement data in a user database.

13. The system of any of aspects 1-12, wherein the diagnostic device comprises an imaging system configured to detect skin conditions.

14. The system of aspect 13, wherein the diagnostic device uses spectral analysis to detect the personal care condition.

15. The system of any of aspects 13-14, wherein the one or more processors are configured to provide information regarding detected skin conditions as updated data and to update the personal care recommendation based on the updated data.

16. The system of any of aspects 13-15 wherein the diagnostic device is integrated into a user interface device that includes the user interface.

17. The system of any of aspects 14-16, wherein the diagnostic device includes one or more of a moisture sensor and a thermal sensor.

18. The system of any of the aspects 1-17, further comprising a user database to store the genetic profile.

19. The system of aspect 18, wherein the user database is encrypted.

20. The system of any of aspect 1-19, wherein the personal care recommendation includes a product recommendation.

21. The system of aspect 20, wherein the product recommendation includes a recommendation of at least one skin care product.

22. The system of any of aspects 20-21, wherein the one or more processors are configured to provide access to an online purchasing system to purchase a product associated with the product recommendation.

23. The system of any of aspects 20-22, wherein the product recommendation is generated to account for a user preference for one or more of cruelty-free products, organic products, or vegan beauty products.

24. The system of any of aspects 1-23, wherein the personal care recommendation includes a recommendation for at least one of timing or sequence for application of personal care operations.

25. The system of any of the preceding aspects, wherein the one or more processors are configured to provide access to further information, including at least one of science information, chemistry information, or expert advice, pertaining to the personal care recommendation.

26. The system of any of the preceding aspects, wherein predicting the personal care condition further includes requesting or receiving input pertaining to lifestyle information, diet information, environmental information pertaining to the user or a location of the user.

27. The system of aspect 26, wherein the input is received from a wearable device or an Internet of Things (IoT) device proximate the user.

28. The system of any of aspects 1-27, further comprising at least one wired or wireless interface to a network, and wherein the one or more processors are configured to provide access to a social media network and wherein the user interface implements functionality to share the personal care recommendation with the social media network.

29. The system of aspect 28, wherein the one or more processors are configured to retrieve data from the social media network pertaining to the personal care recommendation or products similar to products, to update a trained machine learning model.

30. The system of any of aspects 28-29, wherein a trained machine learning model implements natural language processing (NLP) to detect patterns and correlations in data posted on the social media network.

31. The system of any of aspects 28-30, wherein a trained machine learning model receives emotional health as an input from the social media network and generates an updated product recommendation based on the emotional health.

32. The system of any of aspects 1-31, wherein the one or more processors are configured to provide an alert system to alert the user to product updates of products related to the personal care recommendation.

33. The system of any of the preceding aspects, wherein the user interface includes at least one display for providing the user with an augmented reality (AR) simulation of potential impacts of at least one product recommended in the personal care recommendation over time based on the genetic profile.

34. A method of providing a personal care recommendation can comprise assessing a genetic profile of a user; predicting changes in a personal care condition based on at least one of the genetic profile or an output of a diagnostic device; generating the personal care recommendation based on at least one of the personal care condition or the predicted changes in the personal care condition; and providing the personal care recommendation to the user.

35. A non-transitory computer-readable medium storing instructions for providing a personal care recommendation that, when executed on a processor, cause the processor to perform operations including: assessing a genetic profile of a user; predicting changes in a personal care condition based on at least one of the genetic profile and an output of a diagnostic device; generating the personal care recommendation based on at least one of the personal care condition or the predicted changes in the personal care condition; and providing the personal care recommendation to the user.

1. A system for providing a personal care recommendation, the system comprising:
one or more processors;
a diagnostic device coupled to the one or more processors, the diagnostic device configured to detect a personal care condition;
one or more non-transitory memory devices storing computer-readable instructions that, when executed by the one or more processors, cause the one or more processors to:
assess a genetic profile of a user of the system;
identify that the user has at least one characteristic in common with a population or is a member of the population;
use a trained machine learning model to predict changes in the personal care condition based on the genetic profile and on information provided by the diagnostic device, the trained machine learning model being trained to identify at least one skin care product recommended for the user using training data including genetic profiles of the population labeled with skin care products used by members of the population; and
generate the personal care recommendation based on at least one of the personal care condition or predicted changes in the personal care condition; and
a user interface coupled to the one or more processors, the user interface configured to provide the personal care recommendation to the user.

2. The system of claim 1, wherein the one or more processors are configured to assess the genetic profile of the user by:
collecting genetic data from the user; and
identifying genetic markers of the user.

3. The system of claim 2, wherein collecting genetic data comprises receiving data associated with deoxyribonucleic acid (DNA) sample of the user.

4. The system of claim 2, wherein collecting genetic data comprises accessing a genetic testing service.

5. The system of claim 2, wherein collecting the genetic data includes providing an encryption system to protect privacy of the genetic data.

6. The system of claim 2, wherein the genetic markers indicate conditions that affect one or more of skin health, skin care product efficacy, or potential allergic reactions to skin care products.

7.-8. (canceled)

9. The system of claim 1, wherein the one or more processors are further configured to update the trained machine learning model using expanded training data of a second population that is a superset of the population wherein the one or more processors are configured to identify that the user has at least one characteristic in common with the second population or is a member of the second population.

10. The system of claim 1, wherein the one or more processors are further configured to update the trained machine learning model with global datasets to predict personal care conditions that are prevalent among one or more of an ethnic group, a cultural group, or a national group.

11. The system of claim 1, wherein the one or more processors are further configured to update the trained machine learning model based on feedback input by the user.

12. The system of claim 1, wherein the one or more processors are configured to store diagnostic device measurement data in a user database.

13. The system of claim 1, wherein the diagnostic device comprises an imaging system configured to detect skin conditions.

14. The system of claim 13, wherein the diagnostic device uses spectral analysis to detect the personal care condition.

15. The system of claim 13, wherein the one or more processors are configured to provide information regarding detected skin conditions as updated data and to update the personal care recommendation based on the updated data.

16. The system of claim 13, wherein the diagnostic device is integrated into a user interface device that includes the user interface.

17. The system of claim 13, wherein the diagnostic device includes one or more of a moisture sensor and a thermal sensor.

18. The system of claim 1, further comprising a user database to store the genetic profile.

19. The system of claim 18, wherein the user database is encrypted.

20. The system of claim 1, wherein the personal care recommendation includes a product recommendation.

21. The system of claim 20, wherein the product recommendation includes a recommendation of at least one skin care product.

22. The system of claim 20, wherein the one or more processors are configured to provide access to an online purchasing system to purchase a product associated with the product recommendation.

23. The system of claim 20, wherein the product recommendation is generated to account for a user preference for one or more of cruelty-free products, organic products, or vegan beauty products.

24. The system of claim 1, wherein the personal care recommendation includes a recommendation for at least one of timing or sequence for application of personal care operations.

25. The system of claim 1, wherein the one or more processors are configured to provide access to further information, including at least one of science information, chemistry information, or expert advice, pertaining to the personal care recommendation.

26. The system of claim 1, wherein predicting the personal care condition further includes requesting or receiving input pertaining to lifestyle information, diet information, environmental information pertaining to the user or a location of the user.

27. The system of claim 26, wherein the input is received from a wearable device or an Internet of Things (IoT) device proximate the user.

28. The system of claim 1, further comprising at least one wired or wireless interface to a network, and wherein the one or more processors are configured to provide access to a social media network and wherein the user interface implements functionality to share the personal care recommendation with the social media network.

29. The system of claim 28, wherein the one or more processors are configured to retrieve data from the social media network pertaining to the personal care recommendation or products similar to products, to update a trained machine learning model.

30. The system of claim 28, wherein a trained machine learning model implements natural language processing (NLP) to detect patterns and correlations in data posted on the social media network.

31. The system of claim 28, wherein a trained machine learning model receives emotional health as an input from the social media network and generates an updated product recommendation based on the emotional health.

32. The system of claim 1, wherein the one or more processors are configured to provide an alert system to alert the user to product updates of products related to the personal care recommendation.

33. The system of claim 1, wherein the user interface includes at least one display for providing the user with an augmented reality (AR) simulation of potential impacts of at least one product recommended in the personal care recommendation over time based on the genetic profile.

34. A computer-implemented method of providing a personal care recommendation, the method comprising:
assessing a genetic profile of a user;
identifying that the user has at least one characteristic in common with a population or is a member of the population;
using a machine learning model to predict changes in a personal care condition based on at least one of the genetic profile or an output of a diagnostic device, the machine learning model being trained to identify at least one skin care product recommended for the user using training data including genetic profiles of the population labeled with skin care products used by members of the population;
generating the personal care recommendation based on at least one of the personal care condition or the predicted changes in the personal care condition; and
providing the personal care recommendation to the user.

35. A non-transitory computer-readable medium storing instructions for providing a personal care recommendation that, when executed on a processor, cause the processor to perform operations including:
assessing a genetic profile of a user;
identifying that the user has at least one characteristic in common with a population or is a member of the population;
using a machine learning model to predict changes in a personal care condition based on at least one of the genetic profile and an output of a diagnostic device, the machine learning model being trained to identify at least one skin care product recommended for the user using training data including genetic profiles of the population labeled with the skin care products used by members of the population;
generating the personal care recommendation based on at least one of the personal care condition or the predicted changes in the personal care condition; and
providing the personal care recommendation to the user.
